(19) United States
(12) Patent Application Publication
(10) Pub. No.: US 2025/0186633 A1
(43) Pub. Date: Jun. 12, 2025

(54) METHODS FOR IMAGING INTEGRIN EXPRESSION AND FOR IMAGING SITES OF ENDOMETRIOSIS

(71) Applicant: SERAC HEALTHCARE LTD, London (GB)

(72) Inventors: David HAIL, London (GB); Druin BURCH, London (GB)

(21) Appl. No.: 18/843,430

(22) PCT Filed: Mar. 2, 2023
(86) PCT No.: PCT/GB2023/050495

§ 371 Date: Sep. 3, 2024

(30) Foreign Application Priority Data
Mar. 2, 2022 (GB) 2202919.3

(51) Int. Cl. A61K 51/08

(52) U.S. Cl. CPC A61K 51/082; A61K 51/088

(57) ABSTRACT

The present invention relates to methods of imaging sites of endometriosis using the radiopharmaceutical agent ^{99m}-maraciclatide. The technetium-99m radiopharmaceutical is suitably prepared from a non-radioactive kit containing the RGD peptide maraciclatide. Also described are methods of diagnosis, therapy selection and therapy monitoring of endometriosis using the agent. The invention also includes the use of the kit and/or gamma camera or gamma detector in the methods of the invention.

### FIELD OF THE INVENTION

The present invention relates to methods of imaging sites of endometriosis using the radiopharmaceutical agent ^{99m}Tc-maraciclatide. The technetium-99m radiopharmaceutical is suitably prepared from a non-radioactive kit. Also described are methods of diagnosis, therapy selection and therapy monitoring of endometriosis using the agent. The invention also includes the use of the kit and/or gamma camera or gamma detector in the methods of the invention.

### BACKGROUND TO THE INVENTION

The endometrium is the epithelial lining of the cavity of the uterus along with its mucous membrane. The endometrial lining is normally shed once a month during menstruation, and then regrows. Endometriosis is a gynaecological disorder of the female reproductive system, characterised by the presence of abnormal (i.e. ectopic, out of place), proliferating endometrial cells outside the uterus. The mechanism is not fully understood, but is believed to involve attachment of some of the endometrium shed during menstruation. The ectopic growth is commonly found in the pelvis, such as the ovaries or ligaments that support the uterus. Less commonly, the ectopic tissue adheres to the outer surface of the small or large intestines, the ureters, the bladder, the vagina or the lining of the abdominal or chest cavity. Thus, endometriosis manifests itself as: superficial implants in the peritoneum (the folded membrane which covers the digestive organs such as intestines and pancreas); ovarian cysts (endometriomas) or as deep infiltrating lesions located typically in the rectovaginal fascia, uterosacral ligaments and pelvic portions of the urinary or gastrointestinal tracts. More rarely, endometriosis can occur outside the abdomen and pelvis, as in the thorax.

Endometriosis is a benign disease, but can behave like a malignant one by exhibiting abnormal growth and infiltration into normal tissues. Endometriosis is a debilitating condition that is estimated to affect 3 to 10% of the female population of reproductive age, causing pelvic pain, dyspareunia, infertility, depression and a host of other problems. In up to 20% of cases, a type of endometriosis called, “deep infiltrating” or “deeply infiltrative endometriosis” (DIE) occurs deep within the tissue or organ (i.e. at a depth of at least 5 mm)—especially organs that are near the uterus including the bowel and the urinary bladder.

Diagnosis of endometriosis is challenging, with an average of seven years delay between onset of symptoms and eventual diagnosis. Clinical assessment alone is unreliable for making the diagnosis. Current non-invasive techniques such as ultrasound (specifically transvaginal sonography or TVS), MRI and blood tests such as serum CA-125 measurements, have inadequate diagnostic accuracy. Ultrasound and MRI have utility in diagnosing ovarian endometrioma and DIE respectively, but little or no value in the diagnosis of peritoneal endometriosis. Consequently, confirmation of diagnosis typically relies on lesion characterisation via invasive laparoscopic surgery. Laparoscopy with lesion biopsy is the current ‘gold standard’ diagnosis of endometriosis.

U.S. Pat. No. 6,540,980 teaches methods of diagnosing endometriosis using an agent which comprises a binding agent for eosinophil peroxidase coupled to a detectable label, such as a radiolabel, MRI agent or fluorescent label.

U.S. Pat. No. 9,861,711 teaches an in vivo method for the diagnosis of endometriosis using a labelled ligand of anti-Mullerian hormone.

Healy et al, Human Reprod. Update, 4(5), 736-740 (1998) provide a review, which discloses that there is increased endometrial angiogenesis in women with endometriosis vs controls. Healy et al is silent on the imaging of endometriosis, and teach towards investigating drugs which inhibit angiogenesis as potential therapies for endometriosis.

Cosma et al [J.Obstr. Gynaecol., 42(12), 1724-1733 (2016)] studied the use of the PET radiotracer ¹⁸F-estradiol in a PET/CT study of 4 DIE subjects. They reported greater accuracy of detection than MRI, but inability to identify small nodules on the bladder wall. They suggested that the PET/CT imaging should be carried out in the first part of the patient's menstrual cycle, when expression of estrogen is greater with a consequent expected higher probability of endometriotic lesion visualisation.

Fastrez et al [Eur.J.Obst.Gynecol. Reprod.Biol., 212, 69-74 (2017)] disclose the use of a somatostatin receptor-targeting PET radiotracer agent ⁶⁸Ga-labelled dotatate in a pilot clinical study of endometriosis patients. They reported poor sensitivity and specificity in diagnosing DIE recto-vaginal lesions.

Silveira et al [Reprod.Sci., 25(1), 19-25 (2018)] disclose the use of radiotracer ¹⁸F-fluorocholine imaging in a rat model of endometriosis. They suggest that the technique should be evaluated further for the detection of small, superficial endometriosis lesions in humans.

Amartuvshin et al [J.Endomet.Pelv.Pain Dis., 11(4), 194-200 (2019)] disclose imaging studies in a murine model of endometriosis. They argued that vascular endothelial growth factor (VEGF) is essential for the survival and proliferation of endometriosis, and hence used the PET radiotracer ⁶⁴Cu-labelled antibody bevacizumab (an anti-VEGF antibody). They concluded that the tracer may potentially be useful for endometriosis imaging, but no human studies have since been reported.

U.S. Pat. No. 10,259,844 teaches methods of imaging endometriosis based on the peptide Val-Arg-Arg-Ala-Asp-Asn-Arg-Pro-Gly labelled with a detectable marker.

WO 2005/030265 teaches contrast agents for the optical imaging of endometriosis, which comprise a vector for an abnormally-expressed biological target associated with endometriosis, conjugated to an optical reporter moiety. A wide variety of such biological targets is described, including: angiogenesis targets; progesterone receptors; estrogen receptors and folate-binding proteins. WO 2005/030265 does not, however, provide any data as to the effectiveness or otherwise of any of the possible biological approaches described therein.

Velikyan et al [Am.J.Nucl.Med.Mol.Imaging, 8(1), 15-31 (2018)] teach methods for the synthesis of a ⁶⁸Ga-labelled RGD peptide for the non-invasive imaging of angiogenesis. Velikyan et al prepare a chelator conjugate of the bicyclic RGD octapeptide NC100717, where the chelator is DOTA. They report specific uptake in the walls of the uterus in a non-human primate imaging study. They mention that angiogenesis contributes to: cancer, binding diseases, psoriasis, arthritis, endometriosis, multiple sclerosis, and obesity.

WO 03/006491 discloses compounds of Formula (I):

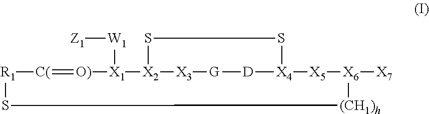

or pharmaceutically acceptable salt thereof
wherein:
G represents glycine
D represents aspartic acid
R₁ represents —(CH₂)ₙ— or —(CH₂)ₙ—C₆H₄— wherein n represents a positive integer 1 to 10,
h represents a positive integer 1 or 2,
X₁ represents an amino acid residue wherein said amino acid possesses a functional side-chain such as an acid or amine,
X₂ and X₄ represent independently an amino acid residue capable of forming a di sulfide bond,
X₃ represents arginine, N-methylarginine or an arginine mimetic,
X₅ represents a hydrophobic amino acid or derivatives thereof,
X₆ represents a thiol-containing amino acid residue,
X₇ is absent or represents a biomodifier moiety,
Z₁ represents an anti-neoplastic agent, a chelating agent or a reporter moiety and
W₁ is absent or represents a spacer moiety.

WO 03/006491 discloses that a preferred chelating moiety has the formula shown, and includes ^{99m}Tc complexes of said chelator conjugate of Formula I therein:

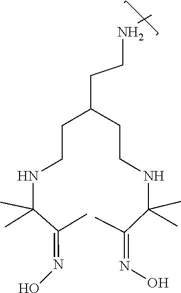 [SMILES: CNCCC(CCNC(C)(C)/C(C)=N\O)CCNC(C)(C)/C(C)=N\O]

WO 03/006491 states that: “Diseases and indications associated with angiogenesis are e.g. different forms of cancer and metastasis, e.g. breast, skin, colorectal, pancreatic, prostate, lung or ovarian cancer. Other diseases and indications are inflammation (e.g. chronic), atherosclerosis, rheumatoid arthritis and gingivitis. Further diseases and indications associated with angiogenesis are arteriovenous malformations, astrocytomas, choriocarcinomas, glioblastomas, gliomas, haemangiomas (childhood, capillary), hepatomas, hyperplastic endometrium, ischemic myocardium, endometriosis, Kaposi sarcoma, macular degeneration, melanoma, neuroblastomas, occluding peripheral artery disease, osteoarthritis, psoriasis, retinopathy (diabetic, proliferative), scleroderma, seminomas and ulcerative colitis.” However, this is a broad list of possibilities and WO 03/006491 does not disclose which agents and ‘reporter moieties’ within the scope would be useful for which medical conditions, and how each of these conditions may be imaged in a way that can be usefully interpreted.

EP 2598175 B1 discloses radiopharmaceutical compositions of the bicyclic RGD peptide maraciclatide, labelled with ^{99m}Tc and stabilised with para-aminobenzoic acid (pABA). Also described are non-radioactive kits containing pABA for the preparation of such radiopharmaceutical compositions.

U.S. Pat. No. 10,729,793 discloses methods of imaging arthritis using radiopharmaceuticals based on chelator conjugates of bicyclic RGD peptides similar to those of Formula I of WO 03/006491 (above). A preferred such radiopharmaceutical for imaging arthritis is ^{99m}Tc-maraciclatide.

### SUMMARY OF THE PRESENT INVENTION

There is a need for a non-invasive imaging method useful in the diagnosis of endometriosis. The present invention provides a non-invasive, radiopharmaceutical imaging method to assist in the diagnosis of endometriosis. The present method avoids the routine need for surgical intervention (such as laparoscopy). It also has a lower radiation dose to the patient than prior art PET agents using positron-emitting radioisotopes such as ¹⁸F or ⁶⁸Ga. ^{99m}Tc-maraciclatide undergoes rapid clearance in vivo, which helps reduce radiation dose to the patient, as well as being favourable for imaging. Since it uses the generator-produced radioisotope ^{99m}Tc, together with a non-radioactive kit for the preparation of ^{99m}Tc-maraciclatide, the radiopharmaceutical itself is expected to be widely-available. Similarly, since ^{99m}Tc is a gamma-emitting radioisotope long-established in nuclear medicine, there is an extensive installed base of suitable gamma cameras for the imaging of the invention. Use of the more ubiquitous ^{99m}Tc radionuclide thus provides broad access to diagnosis, which should shorten significantly the average time between symptom onset and final diagnosis, enabling earlier treatment, hence reducing the need for laparoscopic surgery. The present method also assist in identifying women who do not have active endometriosis, and hence individuals where alternative diagnoses need to be pursued.

In descending order of frequency, endometriosis manifests itself as: superficial peritoneal lesions, endometriomas, deep infiltrating endometriosis (DIE) and extra-pelvic lesions. Unlike some prior art methodology, the present method is applicable to superficial endometriotic lesions, i.e. is not limited to deep infiltrating endometriosis (DIE). Thus, only ovarian endometrioma and deep nodular forms of disease can be detected through ultrasonography and MRI. Ultrasound is also time-consuming, operator-dependent, often uncomfortable or invasive (especially when being performed trans-vaginally, as it is for endometriosis), and unable to detect the commonest forms of the disease. MRI is time-consuming, expensive, extremely limited in its ROI (region of interest) or patient geographic focus, and again unable to detect the commonest forms of the disease. Thus, neither provide the possibility of whole body images, nor imaging of the whole abdomen and pelvis of the subject, nor are they capable of detecting the majority of cases. The present method is capable of rapidly imaging all areas associated with endometriosis (pelvis, abdomen and thorax) within one imaging session without patient discomfort.

The present method is also applicable to peritoneal endometriosis, where ultrasound and MRI have little diagnostic value. The method is not based on estradiol, and hence can be carried out at any stage of the menstrual cycle. In addition, the ability to visualise lesions radiographically permits the monitoring of treatments for endometriosis, as well as the testing of potential new treatments for the condition.

### DETAILED DESCRIPTION OF THE INVENTION

In a first aspect, the present invention provides a method of imaging site(s) of endometriosis in a subject, which comprises prior administration of the radiopharmaceutical ^{99m}Tc-maraciclatide to said subject, followed by extra-uterine imaging of the radioactive emissions from said ^{99m}Tc-maraciclatide in vivo, wherein said site(s) comprise one or more of the following:
(i) abdominal endometriosis;
(ii) peritoneal endometriosis;
(iii) thoracic endometriosis.

The terms “comprising” or “comprises” have their conventional meaning throughout this application and imply that the composition must have the components listed, but that other, unspecified compounds or species may be present in addition. The term ‘comprising’ includes as a preferred subset “consisting essentially of” which means that the composition has the components listed without other compounds or species being present.

The term “subject” refers to an intact mammalian body in vivo, preferably a human female patient, more preferably a human female of reproductive age. For the latter, the present invention is expected to be most useful in the proliferative phases of the menstrual cycle, when the endometrial lining is growing fastest in readiness for the potential implantation of a fertilised egg.

^{99m}Tc is the radioisotope Technetium-99m, which decays with a half-life of 6.02 hours to technetium-99 (⁹⁹Tc). The radioactive decay is accompanied by the emission of a gamma ray with a photon energy of 140 keV that is near ideal for medical imaging. It is these gamma rays that are the “radioactive emissions” of the first aspect. ^{99m}Tc and ^{99m}Tc-radiopharmaceuticals are well known in the art, and ^{99m}Tc is readily available from commercially-available Technetium-99m generators. It can also be produced in cyclotrons by irradiation of ⁹⁹Mo or ¹⁰⁰Mo. ^{99m}Tc radiopharmaceuticals, methods of imaging and associated kits are described in Technetium-99m Pharmaceuticals: Preparation and Quality Control in Nuclear Medicine, I. Zolle (Ed), Springer (2006).

Maraciclatide is the recommended INN (USA Approved Name) for NC100692. The term “maraciclatide” refers to the compound known in the scientific literature as NC100692 [D. Edwards et al, Nucl.Med.Biol., 35, 365-375 (2008)]. The chemical structure of maraciclatide is as follows:

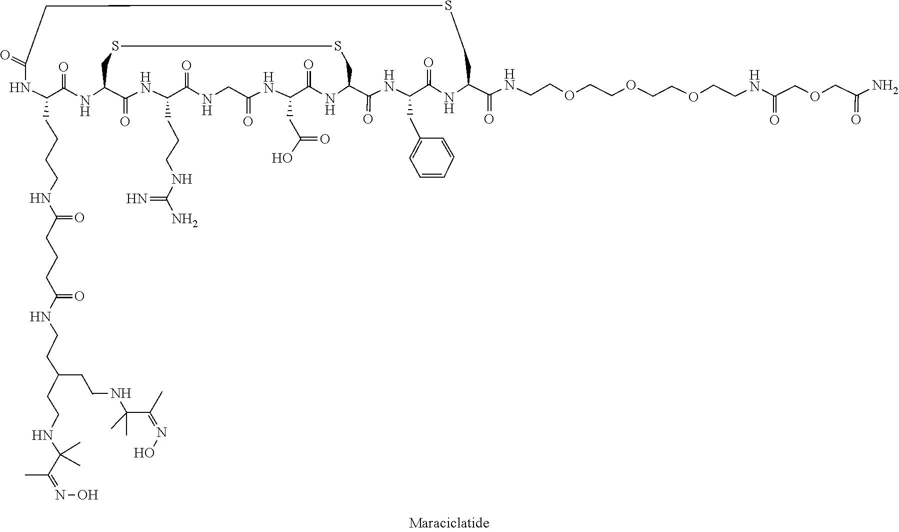

Maraciclatide is a bicyclic RGD (Arg-Gly-Asp) peptide, having conjugated thereto a diaminedioxime chelating agent. The chelating agent forms a metal complex with the technetium radiometal, which is a neutral complex.

Maraciclatide can be used in the free base form, or in the salt form (e.g. the trifluoroacetate). The synthesis of maraciclatide, ^{99m}Tc-maraciclatide and kits for the preparation of ^{99m}Tc-maraciclatide are given in the present Examples.

The term “radiopharmaceutical” has its conventional meaning, and refers to a radioactive compound in a form suitable for in vivo mammalian administration for use in diagnosis or therapy. By the phrase “in a form suitable for mammalian administration” is meant a composition which is sterile, pyrogen-free, lacks compounds which produce toxic or adverse effects, and is formulated at a biocompatible pH (approximately pH 4.0 to 10.5, preferably 6.5 to 9.5 for the agent of the present invention) and physiologically compatible osmolality. Such compositions lack particulates which could risk causing emboli in vivo, and are formulated so that precipitation does not occur on contact with biological fluids (e.g. blood). Such compositions also contain only biologically compatible excipients, and are preferably isotonic.

The ^{99m}Tc-maraciclatide radiopharmaceutical of the present invention is provided in a biocompatible carrier. The “biocompatible carrier” is a fluid, especially a liquid, in which the radiopharmaceutical can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

The radiopharmaceutical composition is suitably provided in a pharmaceutical grade container. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (e.g. to change the headspace gas or degas solutions), and withstand pressure changes such as reductions in pressure without permitting ingress of external atmospheric gases, such as oxygen or water vapour. Preferred multiple dose containers comprise a single bulk vial (e.g. of 6 to 30 cm³ volume) which contains multiple patient doses, whereby single patient doses can thus be withdrawn into clinical grade syringes at various time intervals during the viable lifetime of the preparation to suit the clinical situation.

The radiopharmaceutical composition may also be provided in a syringe. Pre-filled syringes are designed to contain a single human dose, or “unit dose” and are therefore preferably a single-use or other syringe suitable for clinical use.

The term “prior administration” refers to the fact that the ^{99m}Tc-maraciclatide radiopharmaceutical is administered to the subject before the imaging of the first aspect is carried out. Preferably, the ^{99m}Tc-maraciclatide radiopharmaceutical is administered to the subject in a minimally invasive manner, i.e. without a substantial health risk to the mammalian subject even when carried out under professional medical expertise. Such minimally invasive administration is preferably intravenous administration into a peripheral vein of said subject, without the need for local or general anaesthetic.

By the phrase “one or more” of options (i)-(iii) is meant that these can be imaged singly or in combination of 2 thereof, or all 3—either by judicious choice of the field of view and/or region of interest (ROI) of the detector/camera, or by torso or whole body imaging of the subject.

By the term “extra-uterine” is meant all parts of the body lying outside of the body of the uterus and the endometrium. The term “endometrium” refers to the inner epithelial layer of the uterus, and its mucous membrane. Hence extra-uterine endometriosis imaging means radiological visualisation of ectopic endometrial material.

By the term “abdominal endometriosis” is meant the abdomen area of the subject, which has its conventional meaning i.e. the area comprising the stomach, liver, kidneys and intestines of the subject. By the term “pelvis” is meant that portion of the body immediately below the abdomen, forming the lumbar portion of the trunk and containing the bony pelvis, the pelvic floor and pelvic cavity and the perineum. In the female subjects of the invention, this includes the ovaries and the ligaments that support the uterus. The term “pelvic” has its' conventional meaning, and hence “pelvic endometriosis” refers to the area of the pelvis of the subject.

By the term “peritoneal endometriosis” is meant endometriosis which is part of the peritoneal anatomy, specifically its contents (i.e. the small intestine, large intestine and pancreas), or the peritoneum membrane itself which covers these organs.

By the term “superficial endometriotic lesions” is meant endometrial tissue attached to the surface of organs or anatomical structures, as opposed to having a depth of penetration exceeding 5 mm, which is the definition of DIE. By the term “thoracic endometriosis” is meant endometrial tissue in the thorax of the subject. Thoracic endometriosis thus includes the lungs, but also lesions outside the lungs but inside the thorax, i.e. on the inside of the thoracic cavity.

In the method of the first aspect, superficial endometriotic lesions are preferably imaged in combination with abdominal, peritoneal or thoracic endometriosis. The method of the first aspect is most preferably used for the imaging of superficial endometriotic lesions in combination with peritoneal endometriosis, i.e. for the imaging of superficial peritoneal disease.

In the method of the first aspect, all 3 of (i)-(iii) may preferably be imaged together via whole body imaging of said subject. By the term “whole body imaging” is meant imaging of the whole body of the subject in a single imaging procedure.

In the method of the first aspect, the radioactive emissions are preferably detected and processed using: a conventional or high resolution gamma camera; a gamma detector; image processing software or combinations thereof. Conventional gamma cameras or ‘Anger cameras’ typically comprise a sodium iodide scintillator crystal coupled to an array of photomultiplier tubes. In the present invention, the gamma camera is preferably a high resolution gamma camera capable of 360-degree imaging and with sub-millimetre spatial resolution. Gamma camera is most preferably a high resolution gamma camera which comprises a CZT detector. The “CZT detector” is a Cd—Zn—Te or cadmium-zinc-telluride solid state detector, and suitable such cameras equipped with CZT detectors are commercially available as Veriton CT, D-SPECT (Spectrum Dynamics Medical, Israel) or Starguide/Discovery NM/CT 870 CZT, NM 530c (GE Healthcare). The imaging typically commences 1 minute to 36-hours after administration of the radiopharmaceutical of the invention, with image acquisition for 5 to 45 minutes by planar scintigraphy, SPECT or SPECT-CT. Further information on radiopharmaceutical emission detection for medical imaging and/or diagnosis is given in the second aspect (below). The sensitivity of the CZT detector permits effective imaging with lower doses of radiopharmaceutical, thus reducing the radiation burden to the subject. This also permits effective imaging at longer times after administration, for example in instances where it is necessary to wait several half-lives of ^{99m}Tc to allow for background clearance from organs which may otherwise adversely affect the signal-to-background ratio. The CZT detector also provides improved resolution over conventional SPECT imaging.

Some prior art radiotracer imaging agents suffer from background issues. Thus, ¹⁸F-estradiol has high accumulation in the abdominal area and ¹⁸F-choline has high urinary tract accumulation. Both are significant disadvantages in terms of signal-to-background ratio when attempting to image the pelvic region. ^{99m}Tc-maraciclatide exhibits some background uptake in bladder and intestines. Whilst, there is background activity in the peritoneal region, this can be discriminated due to the high level of signal in endometriosis lesions—i.e. the signal-to-background ratio. Any endometrial lesions outside the abdomen (e.g. the lungs), whilst rare, is easily visible due to the low background of ^{99m}Tc-maraciclatide.

In the method of the first aspect, the ^{99m}Tc-maraciclatide radiopharmaceutical is suitably prepared by reaction of maraciclatide with ^{99m}Tc-pertechnetate in the presence of a reducing agent, as is known in the art for technetium radiopharmaceuticals. The ^{99m}Tc-maraciclatide radiopharmaceutical is preferably prepared by reconstitution of a lyophilised, maraciclatide-containing non-radioactive kit with a solution of ^{99m}Tc-pertechnetate. Such a kit preferably comprises:
a) maraciclatide;
b) para-aminobenzoic acid or a salt thereof with a biocompatible cation;
c) a stannous reductant;
d) methylene diphosphonic acid or a salt thereof with a biocompatible cation.

para-Aminobenzoic acid is commercially available, including in pharmaceutical grade purity. Preferably, pharmaceutical grade material is used. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium. Most preferably, the para-aminobenzoic acid salt of the present invention consists essentially of sodium para-aminobenzoate.

By the term “kit” is meant one or more pharmaceutical grade containers, comprising the necessary non-radioactive chemicals to prepare the desired radiopharmaceutical composition, together with operating instructions. The kit is designed to be reconstituted with ^{99m}Tc, especially as pertechnetate, to give a solution suitable for human administration with the minimum of manipulation. The kit of the present invention preferably comprises a lyophilised composition containing all the kit components in a single lyophilised formulation in a single container.

The term “stannous reductant” has its conventional meaning in the field of ^{99m}Tc radiopharmaceuticals and kits, and refers to a salt of Sn²⁺, i.e. tin in the Sn(II) oxidation state. Suitable such salts may be in the hydrated or anhydrous form, and include: stannous chloride, stannous fluoride and stannous tartrate. A preferred such stannous reductant is stannous chloride. The term “methylene diphosphonic acid” has its conventional chemical meaning, and is abbreviated MDP.

The kit is preferably lyophilised and is designed to be reconstituted with sterile ^{99m}Tc-pertechnetate (TcO₄⁻) from a ^{99m}Tc radioisotope generator to give a solution suitable for human administration without further manipulation. The kit preferably further comprises a buffer which comprises a mixture of sodium hydrogen carbonate and anhydrous sodium carbonate.

A most preferred kit formulation is as follows (Example 7):

| Component | Quantity per vial | |
|---|---|---|
| Maraciclatide | 75 | μg |
| Stannous chloride dihydrate | 17.8 | μg |
| Methylene diphosphonic acid (MDP) | 90 | μg |
| Para-aminobenzoic acid | 200 | μg |
| (pABA), sodium salt | | |
| Sodium hydrogen carbonate | 1800 | μg |
| Sodium carbonate anhydrous | 630 | μg |

The method of imaging of the first aspect is useful in the diagnosis of endometriosis, but is not the complete diagnosis. That is because the radiopharmaceutical images obtained would need one or more of: (a) comparison with normal, i.e. disease-free images; (b) interpretation by a clinical expert, or (c) analysis by suitable computerised algorithms or artificial intelligence—alone or in combinations thereof. That diagnostic method is addressed in the second aspect below.

In a second aspect, the present invention provides a method of diagnosis of endometriosis which comprises the method of imaging as defined in the first aspect.

Preferred aspects of the sites of endometriosis, subject, radioactive emissions detection; the ^{99m}Tc-maraciclatide radiopharmaceutical; the method of preparation of ^{99m}Tc-maraciclatide and non-radioactive kit in the second aspect are as described in the first aspect (above).

The method of diagnosis of the second aspect includes the step of administration of the ^{99m}Tc-maraciclatide radiopharmaceutical to the subject.

The imaging of the first aspect provides medical images. The data from the imaging of the subject using a suitable gamma camera or gamma detector is processed by algorithms as is known in the art to produce medical images. Gamma camera, gamma detectors and image processing is reviewed in: Handbook of Nuclear Medicine and Molecular Imaging for Physicists, M. Ljungberg (Ed), 3 volume set, CRC Press (2021). Image interpretation for a given subject is then achieved by options A and B:

### Option A

(i) review of the images by a radiologist;
(ii) qualitative or quantification scoring of radiopharmaceutical uptake in a region of interest (ROI);
(iii) decision by the radiologist or clinical team as to whether to proceed to laparoscopic surgery or instigate a course of treatment or rule out endometriosis from the diagnosis, and pursue alternative avenues.

### Option B

(i) image analysis using one or more machine learning derived algorithms to highlight lesions in a ROI;
(ii) review of the images by a radiologist;
(iii) decision by radiologist or clinical team as to whether to proceed to laparoscopic surgery or instigate a course of treatment or rule out endometriosis from the diagnosis, and pursue alternative avenues.

In both options A and B, the radiologist may optionally use reference to one or more normal scans. By the term “normal scan” is meant at least one reference image using the same radiopharmaceutical, and preferably the same gamma camera, means of detection and image processing methodology in a different subject, where no endometriosis was found. More than one such normal scan may be used as a library of images, to assist with normal variation in images. Thus, laparoscopic surgery when utilised, would be to obtain a confirmatory diagnosis, especially when used in conjunction with biopsy which would permits clinical characterisation of the lesion, and thus assist in the determination of subsequent patient management or treatment.

In a third aspect, the present invention provides a method of determination of therapy of endometriosis in a subject, which comprises the method of imaging of the first aspect or the method of diagnosis of the second aspect.

Preferred aspects of the sites of endometriosis, subject, radioactive emissions detection, the ^{99m}Tc-maraciclatide radiopharmaceutical, method of preparation of ^{99m}Tc-maraciclatide and non-radioactive kit in the third aspect are as described in the first aspect (above). Preferred aspects of the method of diagnosis in the third aspect are as described in the second aspect.

In this aspect, the imaging of the first aspect may confirm the site(s) of endometriotic tissue. That in itself may be enough information to help determine subsequent patient management or treatment. In other circumstances, the method of diagnosis of the second aspect is necessary. Knowledge of the site of disease may help determine the subsequent treatment or therapy-either surgical or medication or combinations thereof. The treatment of endometriosis has been reviewed by K. T. Zondervan et al [New Eng.J.Med., 383, 1246-1256 (2020)].

In a fourth aspect, the present invention provides a method of monitoring of a therapy of endometriosis in a subject, which comprises the method of imaging of the first aspect or the method of diagnosis of the second aspect.

Preferred aspects of the sites of endometriosis, subject, radioactive emissions detection, method of preparation of ^{99m}Tc-maraciclatide and non-radioactive kit in the fourth aspect are as described in the first aspect (above). Preferred aspects of the method of diagnosis in the fourth aspect are as described in the second aspect (above).

By the term “monitoring” is meant carrying out multiple imaging of the same subject before and at chosen time intervals during a course of therapy. Comparison of these images then provides the clinician with information as to whether the therapy is reducing the degree of endometriosis in the ROI for said subject or whether the attempted therapy is proving ineffective by allowing existing lesions to progress, or new lesions to develop. The method of information or diagnosis of the invention is thus expected to help determine whether a given course of therapy is proving successful for the individual subject. If a positive result is found, then later images may be used to confirm the progression of therapy. If negative, then alternative therapies may be chosen and a new monitoring as per this fourth aspect of the invention initiated.

In a fifth aspect, the present invention provides a non-radioactive kit for the preparation of ^{99m}Tc-maraciclatide for use in one or more of the following:
(i) the method of imaging of the first aspect,
(ii) the method of diagnosis of the second aspect;
(iii) the method of determination of therapy of the third aspect;
(iv) the method of monitoring of therapy of the fourth aspect;
   wherein said non-radioactive kit is as defined in the first aspect.

This fifth aspect also includes the use of said non-radioactive kit in one or more of the methods (i)-(iv).

Preferred aspects of the sites of endometriosis, subject, radioactive emissions detection, method of preparation of ^{99m}Tc-maraciclatide and non-radioactive kit in the fifth aspect are as described in the first aspect (above).

In the fifth aspect, the kit is used in a method of preparation of the ^{99m}Tc-maraciclatide radiopharmaceutical, which is then used in one or more of the methods (i)-(iv). Hence, within the scope of this aspect are any of the methods (i)-(iv) where the non-radioactive kit is used to provide the maraciclatide and/or ^{99m}Tc-maraciclatide.

In a sixth aspect, the present invention provides the use of a gamma camera, gamma detector and/or image processing software in one or more of the following:
(i) the method of imaging of the first aspect,
(ii) the method of diagnosis of the second aspect;
(iii) the method of determination of therapy of the third aspect;
(iv) the method of monitoring of therapy of the fourth aspect;

Preferred aspects of the sites of endometriosis, subject, radioactive emissions detection, method of preparation of ^{99m}Tc-maraciclatide and non-radioactive kit in the sixth aspect are as described in the first aspect (above).

In the sixth aspect, the phrase “image processing software” includes algorithms, in particular artificial intelligence algorithms-especially machine learning adapted to work with ^{99m}Tc-maraciclatide images. Gamma camera, gamma detectors and such image processing is reviewed in: Handbook of Nuclear Medicine and Molecular Imaging for Physicists, M. Ljungberg (Ed), 3 volume set, CRC Press (2021). Artificial intelligence in medical imaging has been reviewed by Seah et al

[Br.J.Radiol., 94(1126), 20210406 (2021)]. Machine learning in medical imaging has been described by T. Sadad et al [Curr.Med. Imaging, 17(6), 686-694 (2021)].

In a further aspect, the present invention provides the radiopharmaceutical ^{99m}Tc-maraciclatide for use in an in vivo imaging diagnostic method of extra-uterine endometriosis in a subject, wherein said endometriosis comprises one of the following sites of endometriosis, or combinations thereof:
(i) abdominal endometriosis;
(ii) peritoneal endometriosis;
(iii) thoracic endometriosis.

In this further aspect, the definitions of terms and preferred embodiments thereof are as described in the first and second aspects (above).

The invention is illustrated by the non-limiting Examples detailed below. Examples 1 to 3 provide the synthesis of Chelator 1 (also sometimes called carba-Pn216) of the invention. Example 4 provides the synthesis of Chelator 1A of the invention—an active ester-functionalised version of Chelator 1. Example 5 provides the synthesis of cyclic peptides of the invention and chelator conjugation. Example 6 provides the synthesis of maraciclatide. Example 7 provides the preparation of a maraciclatide lyophilised kit. Example 8 provides the method of reconstituting the kit to obtain the ^{99m}Tc-maraciclatide radiopharmaceutical. Example 9 provides biological data which supports the utility of ^{99m}Tc-maraciclatide for endometriosis tissue uptake, since uptake and retention of ^{99m}Tc-maraciclatide was demonstrated in 3 experimental models.

### DESCRIPTION OF THE FIGURES

FIG. 1 compares the uptake of ^{99m}Tc-maraciclatide and the scrambled control peptide ^{99m}Tc-NC100667 in a rat model of endometriosis. Data is expressed as % id/g and Relative Concentration and shown as Mean±SD; *=p<0.05 endometrial lesions at 2 hours post injection. FIG. 1 shows that ^{99m}Tc-maraciclatide (expressed as % id/g) exhibited a 7-fold greater uptake and retention in endometrial lesions compared to the scrambled control peptide ^{99m}Tc-NC100667. This shows that the endometrial uptake was specific, since it requires the particular chemical structure of ^{99m}Tc-maraciclatide.

FIG. 2 demonstrates ^{99m}Tc-maraciclatide uptake in the same rat endometrial model. Endometrial lesion angiogenesis was measured via micro-vessel density (MVD). Data are expressed as average (a-MVD) and highest (h-MVD) and shown as Mean±SD; *=p<0.05. Uptake in the endometrial lesions was related to the level of angiogenesis (expressed as MVD) and higher compared to control lesions of abdominal fat. This shows that the ^{99m}Tc-maraciclatide uptake was specific for endometrial tissue.

FIG. 3 shows the uptake of ^{99m}Tc-maraciclatide in the Isogenic mouse endometrial lesion at 1, 2 or 3 weeks post implantation (2 hours post injection). Data is expressed as % id/g and shown as Mean±SD; *=p<0.05. A time course experiment was carried out using the isogenic mouse model at one, two or three weeks post uterine horn inoculation. Uptake of ^{99m}Tc-maraciclatide was greatest at one week post uterine horn inoculation. Subsequent biodistribution studies therefore examined the uptake of ^{99m}Tc-maraciclatide at the one week time point.

FIG. 4 demonstrates that the initial uptake of ^{99m}Tc-maraciclatide at 5 minutes post-injection in the lesion was 5.6% id/g, decreasing over time to 3.6% id/g, with 62% of the initial uptake of ^{99m}Tc-maraciclatide retained at 120 minutes post injection. For comparison, ovaries and uterine horns had retention of ˜35%. This suggests that ^{99m}Tc-maraciclatide was specifically retained within the lesion to a greater extent than normal tissue. Furthermore, the ratio of endometriotic lesion to blood and muscle reached 4.0 and 8.3 respectively at 120 minutes.

### ABBREVIATIONS

Conventional single letter or 3-letter amino acid abbreviations are used.

Ac: Acetyl.

Boc: tert-Butyloxycarbonyl.

tBu: tertiary-butyl.

DIE: deep infiltrating endometriosis.

DMF: Dimethylformamide.

DMSO: Dimethylsulfoxide.

Fmoc: 9-Fluorenylmethoxycarbonyl.

HATU: O-(7-Azabenzotriazol-1-yl)-N,N,N′,N′-tetramethyluronium hexafluorophosphate.

HBTU: O-Benzotriazol-1-yl-N,N,N′,N′-tetramethyluronium hexafluorophosphate

HPLC: High performance liquid chromatography.

MDP: methylene diphosphonic acid.

MRI: Magnetic Resonance Imaging.

NMM: N-Methylmorpholine.

pABA: para-amino-benzoic acid sodium salt

PBS: Phosphate-buffered saline

PEG: polyethyleneglycol, repeat units of (OCH₂CH₂)ₙ, where n is an integer,

PET: positron emission tomography.

RCP: radiochemical purity.

ROI: region of interest.

RP-HPLC: reversed-phase HPLC.

SPECT: Single Photon Emission Computer Tomography.

TFA: Trifluoroacetic acid

THF: Tetrahydrofuran.

TIS: Triisopropylsilane

TLC: thin layer chromatography

Trt: Trityl.

Compounds of the Invention.

| Compound | Structure |
|---|---|
| Chelator 1 | 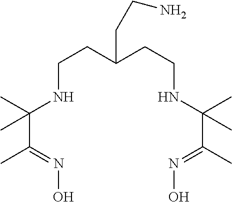 [SMILES: C/C(=N\O)C(C)(C)NCCC(CCN)CCNC(C)(C)/C(C)=N/O] |
| Chelator 1A | 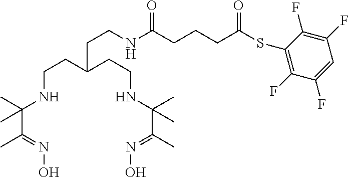 [SMILES: C/C(=N\O)C(C)(C)NCCC(CCNC(=O)CCCC(=O)Sc1c(F)c(F)cc(F)c1F)CCNC(C)(C)/C(C)=N/O] |

### Example 1: Synthesis of 1,1,1-Tris(2-aminoethyl)methane

Step 1(a): 3(methoxycarbonylmethylene)glutaric acid dimethylester.

Carbomethoxymethylenetriphenylphosphorane (167 g, 0.5 mol) in toluene (600 ml) was treated with dimethyl 3-oxoglutarate (87 g, 0.5 mol) and the reaction heated to 100° C. on an oil bath at 120° C. under an atmosphere of nitrogen for 36 h. The reaction was then concentrated in vacuo and the oily residue triturated with 40/60 petrol ether/diethylether (1:1, 600 ml). Triphenylphosphine oxide precipitated out and the supernatant liquid was decanted/filtered off. The residue on evaporation in vacuo was Kugelrohr distilled under high vacuum Bpt (oven temperature 180-200° C. at 0.2 torr) to give 3-(methoxycarbonylmethylene)glutaric acid dimethylester (89.08 g, 53%).

NMR ¹H(CDCl₃): δ 3.31 (2H, s, CH₂), 3.7 (9H, s, 3xOCH₃), 3.87 (2H, s, CH₂), 5.79 (1H, s, ═CH,) ppm.

NMR ¹³C(CDCl₃), δ 36.56,CH₃, 48.7, 2xCH₃, 52.09 and 52.5 (2xCH₂); 122.3 and 146.16 C═CH; 165.9, 170.0 and 170.5 3xCOO ppm.

Step 1(b): Hydrogenation of 3-(methoxycarbonylmethylene)glutaric acid dimethylester.

3-(Methoxycarbonylmethylene) glutaric acid dimethylester (89 g, 267 mmol) in methanol (200 ml) was shaken with (10% palladium on charcoal: 50% water) (9 g) under an atmosphere of hydrogen gas (3.5 bar) for 30 h. The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3xCH₂), 2.78 (1H, hextet, J=8 Hz CH,) 3.7 (9H, s, 3xCH₃).

NMR ¹³C(CDCl₃), δ 28.6, CH; 37.50, 3xCH₃; 51.6, 3xCH₂; 172.28, 3xCOO.

### Step 1(c): Reduction and Esterification of Trimethyl Ester to the Triacetate.

Under an atmosphere of nitrogen in a 3 necked 2 L round bottomed flask lithium aluminium hydride (20 g, 588 mmol) in THF (400 ml) was treated cautiously with tris(methyloxycarbonylmethyl)methane (40 g, 212 mmol) in THF (200 ml) over 1 h. A strongly exothermic reaction occurred, causing the solvent to reflux strongly. The reaction was heated on an oil bath at 90° C. at reflux for 3 days. The reaction was quenched by the cautious dropwise addition of acetic acid (100 ml) until the evolution of hydrogen ceased. The stirred reaction mixture was cautiously treated with acetic anhydride solution (500 ml) at such a rate as to cause gentle reflux. The flask was equipped for distillation and stirred and then heating at 90° C. (oil bath temperature) to distil out the THF. A further portion of acetic anhydride (300 ml) was added, the reaction returned to reflux configuration and stirred and heated in an oil bath at 140° C. for 5 h. The reaction was allowed to cool and filtered. The aluminium oxide precipitate was washed with ethyl acetate and the combined filtrates concentrated on a rotary evaporator at a water bath temperature of 50° C. in vacuo (5 mmHg) to afford an oil. The oil was taken up in ethyl acetate (500 ml) and washed with saturated aqueous potassium carbonate solution. The ethyl acetate solution was separated, dried over sodium sulfate, and concentrated in vacuo to afford an oil. The oil was Kugelrohr distilled in high vacuum to give tris(2-acetoxyethyl)methane (45.3 g, 95.9%) as an oil. Bp. 220° C. at 0.1 mmHg.

NMR ¹H(CDCl₃), δ 1.66 (7H, m, 3xCH₂, CH), 2.08 (1H, s, 3xCH₃); 4.1 (6H, t, 3xCH₂O).

NMR ¹³C(CDCl₃), δ 20.9, CH₃; 29.34, CH; 32.17, CH₂; 62.15, CH₂O; 171, CO.

Step 1(d): Removal of Acetate Groups from the Triacetate.

Tris(2-acetoxyethyl)methane (45.3 g, 165 mM) in methanol (200 ml) and 880 ammonia (100 ml) was heated on an oil bath at 80° C. for 2 days. The reaction was treated with a further portion of 880 ammonia (50 ml) and heated at 80° C. in an oil bath for 24 h. A further portion of 880 ammonia (50 ml) was added and the reaction heated at 80° C. for 24 h. The reaction was then concentrated in vacuo to remove all solvents to give an oil. This was taken up into 880 ammonia (150 ml) and heated at 80° C. for 24 h. The reaction was then concentrated in vacuo to remove all solvents to give an oil. Kugelrohr distillation gave acetamide bp 170-180 0.2 mm. The bulbs containing the acetamide were washed clean and the distillation continued. Tris(2-hydroxyethyl)methane (22.53g, 92%) distilled at bp 220° C. 0.2 mm.

NMR ¹H(CDCl₃), δ 1.45 (6H, q, 3xCH₂), 2.2 (1H, quintet, CH); 3.7 (6H, t 3xCH₂OH); 5.5 (3H, brs, 3xOH).

NMR ¹³C(CDCl₃), δ 22.13, CH; 33.95, 3xCH₂; 57.8, 3xCH₂OH.

Step 1(e): Conversion of the Triol to the tris(methanesulfonate).

To an stirred ice-cooled solution of tris(2-hydroxyethyl)methane (10 g, 0.0676 mol) in dichloromethane (50 ml) was slowly dripped a solution of methanesulfonyl chloride (40 g, 0.349 mol) in dichloromethane (50 ml) under nitrogen at such a rate that the temperature did not rise above 15° C. Pyridine (21.4 g, 0.27 mol, 4 eq) dissolved in dichloromethane (50 ml) was then added drop-wise at such a rate that the temperature did not rise above 15° C., exothermic reaction. The reaction was left to stir at room temperature for 24 h and then treated with 5N hydrochloric acid solution (80 ml) and the layers separated. The aqueous layer was extracted with further dichloromethane (50 ml) and the organic extracts combined, dried over sodium sulfate, filtered and concentrated in vacuo to give tris[2-(methylsulfonyloxy)ethyl]methane contaminated with excess methanesulfonyl chloride. The theoretical yield was 25.8 g.

NMR ¹H(CDCl₃), δ 4.3 (6H, t, 2xCH₂), 3.0 (9H, s, 3xCH₃), 2 (1H, hextet, CH), 1.85 (6H, q, 3×CH₂).

Step 1(f): Preparation of 1,1,1-tris(2-azidoethyl)methane.

A stirred solution of tris[2-(methylsulfonyloxy)ethyl]methane [from Step 1(e), contaminated with excess methylsulfonyl chloride] (25.8 g, 67 mmol, theoretical) in dry DMF (250 ml) under nitrogen was treated with sodium azide (30.7 g, 0.47 mol) portion-wise over 15 minutes. An exotherm was observed and the reaction was cooled on an ice bath. After 30 minutes, the reaction mixture was heated on an oil bath at 50° C. for 24 h. The reaction became brown in colour. The reaction was allowed to cool, treated with dilute potassium carbonate solution (200 ml) and extracted three times with 40/60 petrol ether/diethylether 10:1 (3×150 ml). The organic extracts were washed with water (2×150 ml), dried over sodium sulfate and filtered. Ethanol (200 ml) was added to the petrol/ether solution to keep the triazide in solution and the volume reduced in vacuo to no less than 200 ml. Ethanol (200 ml) was added and reconcentrated in vacuo to remove the last traces of petrol leaving no less than 200 ml of ethanolic solution. The ethanol solution of triazide was used directly in Step 1(g).

CARE: DO NOT REMOVE ALL THE SOLVENT AS THE AZIDE IS POTENTIALLY EXPLOSIVE AND SHOULD BE KEPT IN DILUTE SOLUTION AT ALL TIMES.

Less than 0.2 ml of the solution was evaporated in vacuo to remove the ethanol and an NMR run on this small sample: NMR ¹H(CDCl₃), δ 3.35 (6H, t, 3xCH₂), 1.8 (1H, septet, CH,), 1.6 (6H, q, 3xCH₂).

Step 1(g): Preparation of 1,1,1-tris(2-aminoethyl)methane.

Tris(2-azidoethyl)methane (15.06 g, 0.0676 mol), (assuming 100% yield from previous reaction) in ethanol (200 ml) was treated with 10% palladium on charcoal (2 g, 50% water) and hydrogenated for 12 h. The reaction vessel was evacuated every 2 hours to remove nitrogen evolved from the reaction and refilled with hydrogen. A sample was taken for NMR analysis to confirm complete conversion of the triazide to the triamine.

Caution: unreduced azide could explode on distillation. The reaction was filtered through a celite pad to remove the catalyst and concentrated in vacuo to give tris(2-aminoethyl)methane as an oil. This was further purified by Kugelrohr distillation bp. 180-200° C. at 0.4 mm/Hg to give a colourless oil (8.1 g, 82.7% overall yield).

NMR ¹H(CDCl₃), δ 2.72 (6H, t, 3xCH₂N), 1.41 (H, septet, CH), 1.39 (6H, q, 3xCH₂).

NMR ¹³C(CDCl₃), δ 39.8 (CH₂NH₂), 38.2 (CH₂), 31.0 (CH).

### Example 2: Preparation of 3-Chloro-3-methyl-2-nitrosobutane

A mixture of 2-methylbut-2-ene (147 ml, 1.4 mol) and isoamyl nitrite (156 ml, 1.16 mol) was cooled to −30° C. in a bath of cardice and methanol and vigorously stirred with an overhead air stirrer and treated dropwise with concentrated hydrochloric acid (140 ml, 1.68 mol) at such a rate that the temperature was maintained below −20° C. This requires about 1 h as there is a significant exotherm and care must be taken to prevent overheating. Ethanol (100 ml) was added to reduce the viscosity of the slurry that had formed at the end of the addition and the reaction stirred at −20 to −10° C. for a further 2 h to complete the reaction. The precipitate was collected by filtration under vacuum and washed with 4×30 ml of cold (−20° C.) ethanol and 100 ml of ice cold water, and dried in vacuo to give 3-chloro-3-methyl-2-nitrosobutane as a white solid. The ethanol filtrate and washings were combined and diluted with water (200 ml) and cooled and allowed to stand for 1 h at −10° C. when a further crop of 3-chloro-3-methyl-2-nitrosobutane crystallised out. The precipitate was collected by filtration and washed with the minimum of water and dried in vacuo to give a total yield of 3-chloro-3-methyl-2-nitrosobutane (115 g 0.85 mol, 73%) >98% pure by NMR.

NMR ¹H(CDCl₃), As a mixture of isomers (isomer1, 90%) 1.5 d, (2H, CH₃), 1.65 d, (4H, 2xCH₃), 5.85, q, and 5.95, q, together 1H. (isomer2, 10%), 1.76 s, (6H, 2xCH₃), 2.07 (3H, CH₃).

### Example 3: Synthesis of bis[N-(1,1-dimethyl-2-N-hydroxyimine propyl)2-aminoethyl]-(2-aminoethyl)methane (Chelator 1)

To a solution of tris(2-aminoethyl)methane (Example 1; 4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (Example 2; 7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using PRP reverse phase column in a gradient of 7.5-75% acetonitrile in 3% aqueous ammonia. The reaction was concentrated in vacuo to remove the ethanol and re-suspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24 (6H, s, 2xCH₃), 1.3 (6H, s, 2xCH₃), 1.25-1.75 (7H, m, 3xCH₂,CH), (3H, s, 2xCH₂), 2.58 (4H, m, CH₂N), 2.88 (2H, t CH₂N), 5.0 (6H, s, NH₂, 2xNH, 2xOH).

NMR ¹H((CD₃)₂SO) δ 1.1 4xCH; 1.29, 3xCH₂; 2.1 (4H, t, 2xCH₂);

NMR ¹³C((CD₃)₂SO), δ 9.0 (4xCH₃), 25.8 (2xCH₃), 31.0 2xCH₂, 34.6 CH₂, 56.8 2xCH₂N; 160.3, C═N.

HPLC conditions: flow rate 8 ml/min using a 25 mm PRP column [A=3% ammonia solution (sp.gr=0.88)/water; B=Acetonitrile].

| | Time (min) | 0 | 15 | 20 | 22 | 30 |
|---|---|---|---|---|---|---|
| Gradient | % B | 7.5 | 75.0 | 75.0 | 7.5 | 7.5 |

Load 3 ml of aqueous solution per run, and collect in a time window of 12.5-13.5 min.

### Example 4: Synthesis of Tetrafluorothiophenyl Ester of Chelator 1-Glutaric Acid (Chelator 1A)

### (Step 4a) Synthesis of [Chelator 1]-Glutaric Acid Intermediate.

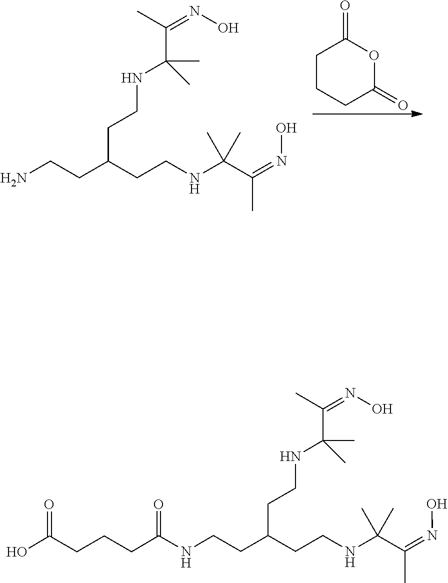 [SMILES: C/C(=N/O)C(C)(C)NCCC(CCN)CCNC(C)(C)/C(C)=N\O ; O=C1CCCC(=O)O1 ; C/C(=N/O)C(C)(C)NCCC(CCNC(=O)CCCC(=O)O)CCNC(C)(C)/C(C)=N\O]

Chelator 1 (100 mg, 0.29 mmol) was dissolved in DMF (10 ml) and glutaric anhydride (33 mg, 0.29 mmol) added by portions with stirring. The reaction was stirred for 23 hours to afford complete conversion to the desired product. The pure acid was obtained following RP-HPLC in good yield.

### (Step 4b) Synthesis of Chelator 1A.

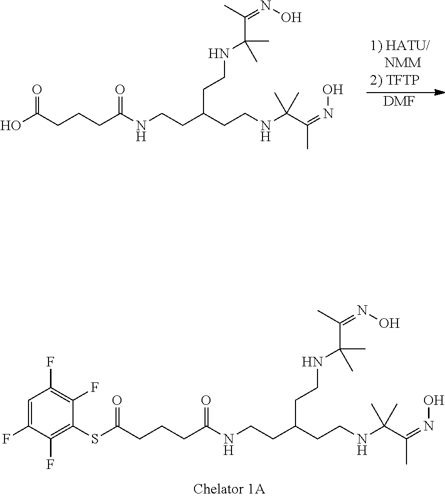

To [Chelator 1]-glutaric acid (from Step 4a; 300 mg, 0.66 mmol) in DMF (2 ml) was added HATU (249 mg, 0.66 mmol) and NMM (132 μL, 1.32 mmol). The mixture was stirred for 5 minutes then tetrafluorothiophenol (0.66 mmol, 119 mg) was added. The solution was stirred for 10 minutes then the reaction mixture was diluted with 20% acetonitrile/water (8 ml) and the product purified by RP-HPLC yielding 110 mg of the desired product following freeze-drying.

### Example 5: Synthesis of Disulfide [Cys²⁻⁶] Thioether Cyclo[CH₂CO-Lys(Chelator 1-Glutaryl)-Cys²-Arg-Gly-Asp-Cys⁶-Phe-Cys]-NH₂.

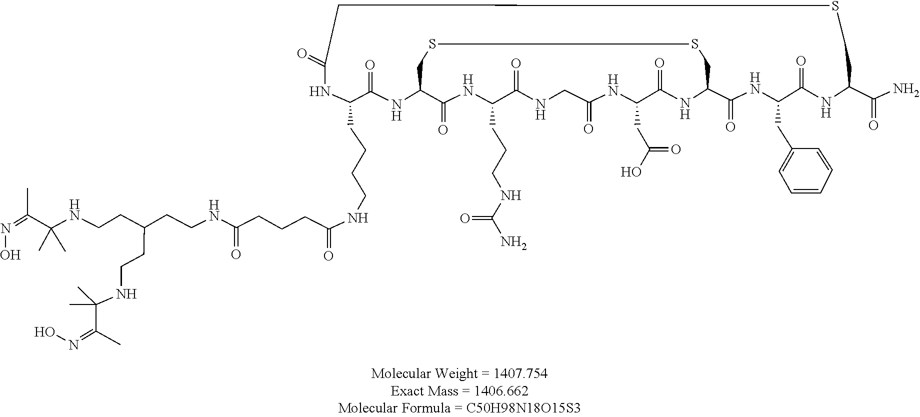

(Step 5a) Synthesis of ClCH₂CO-Lys-Cys(tBu)-Arg-Gly-Asp-Cys(tBu)-Phe-Cys-NH₂

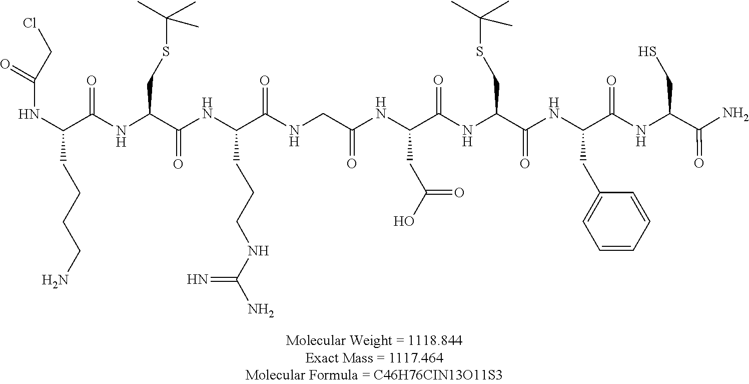 [SMILES: CC(C)(C)SC[C@H](NC(=O)[C@H](CCCCN)NC(=O)CCl)C(=O)N[C@@H](CCCNC(=N)N)C(=O)NCC(=O)N[C@@H](CC(=O)O)C(=O)N[C@@H](CSC(C)(C)C)C(=O)N[C@@H](Cc1ccccc1)C(=O)N[C@@H](CS)C(N)=O]

The peptide was synthesised on an ABI 433A automatic peptide synthesiser starting with Rink Amide AM resin on a 0.25 mmol scale using 1 mmol amino acid cartridges. The amino acids were pre-activated using HBTU before coupling. N-terminal amine groups were chloroacetylated using a solution of chloroacetic anhydride in DMF for 30 min. The simultaneous removal of peptide and side-chain protecting groups (except tBu) from the resin was carried out in TFA containing TIS (5%), H₂O (5%) and phenol (2.5%) for two hours. After work-up 295 mg of crude peptide was obtained (Analytical HPLC: Gradient, 5-50% B over 10 min where A=H₂/0.1% TFA and B=CH₃CN/0.1% TFA; column, Phenomenex Luna 3μ C18 (2) 50×4.6 mm; flow, 2 ml/min; detection, UV 214 nm; product retention time, 6.42 min). Further product characterisation was carried out using mass spectrometry: Expected, M+H at 1118.5, found, at 1118.6).

(Step 5b) Synthesis of Thioether Cyclo[CH₂CO-Lys-Cys(tBu)-Arg-Gly-Asp-Cys(tBu)-Phe-Cys]-NH₂.

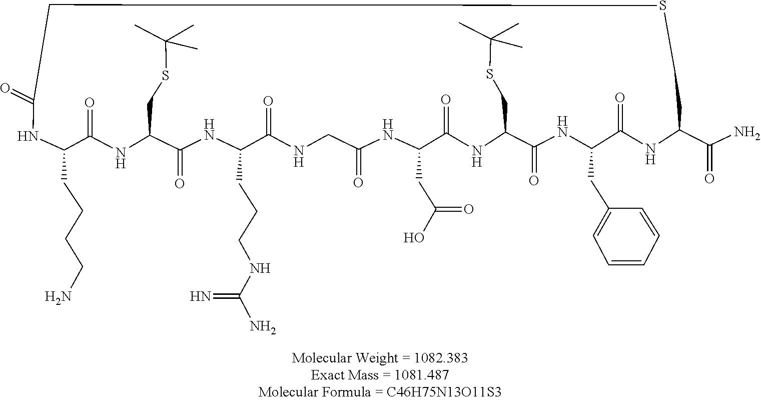 [SMILES: CC(C)(C)SC[C@@H]1NC(=O)[C@H](CCCCN)NC(=O)CSC[C@@H](C(N)=O)NC(=O)[C@H](Cc2ccccc2)NC(=O)[C@H](CSC(C)(C)C)NC(=O)[C@H](CC(=O)O)NC(=O)CNC(=O)[C@H](CCCNC(=N)N)NC1=O]

295 mg of ClCH₂CO-Lys-Cys(tBu)-Arg-Gly-Asp-Cys(tBu)-Phe-Cys-NH₂ was dissolved in water/acetonitrile. The mixture was adjusted to pH 8 with ammonia solution and stirred for 16 hours. After work-up 217 mg of crude peptide was obtained (Analytical HPLC: Gradient, 5-50% B over 10 min where A=H₂O/0.1% TFA and B=CH₃CN/0.1% TFA; column, Phenomenex Luna 3β C18 (2) 50×4.6 mm; flow, 2 ml/min; detection, UV 214 nm; product retention time, 6.18 min). Further product characterisation was carried out using mass spectrometry: Expected, M+H at 1882.5, found, at 1882.6).

(Step 5c) Synthesis of Disulfide [Cys²⁻⁶] Thioether Cyclo[CH₂CO-Lys-Cys²-Arg-Gly-Asp-Cys⁶-Phe-Cys]-NH₂.

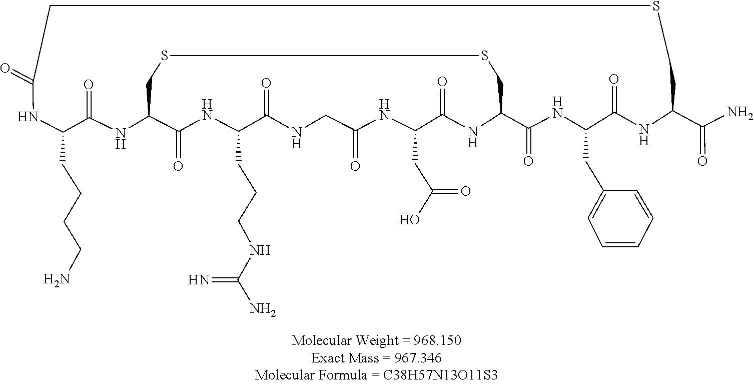

217 mg of thioether cyclo[CH₂CO-Lys-Cys(tBu)-Arg-Gly-Asp-Cys(tBu)-Phe-Cys]-NH₂ was treated with a solution of anisole (500 μL), DMSO (2 ml) and TFA (100 ml) for 60 min following which the TFA was removed in vacuo and the peptide precipitated by the addition of diethyl ether. Purification by preparative HPLC (Phenomenex Luna 10μ C18 (2) 250×50 mm column) of the crude material (202 mg) was carried out using 0-30% B, where A=H₂O/0.1% TFA and B=CH₃CN/0.1% TFA, over 60 min at a flow rate of 50 ml/min. After lyophilisation 112 mg of pure material was obtained (Analytical HPLC: Gradient, 5-50% B over 10 min where A=H₂O/0.1% TFA and B=CH₃CN/0.1% TFA; column, Phenomenex Luna 3μ C18 (2) 50×4.6 mm; flow, 2 ml/min; detection, UV 214 nm; product retention time, 5.50 min). Further product_characterisation was carried out using mass spectrometry: Expected, M+H at 968, found, at 971).

(Step 5d) Synthesis of Disulfide [Cys²⁻⁶] Thioether Cyclo[CH₂CO-Lys(Chelator 1-glutaryl)-Cys²-Arg-Gly-Asp-Cys⁶-Phe-Cys]-NH₂.

9.7 mg of disulfide[Cys²⁻⁶] thioether cyclo[CH₂CO-Lys-Cys-Arg-Gly-Asp-Cys-Phe-Cys]

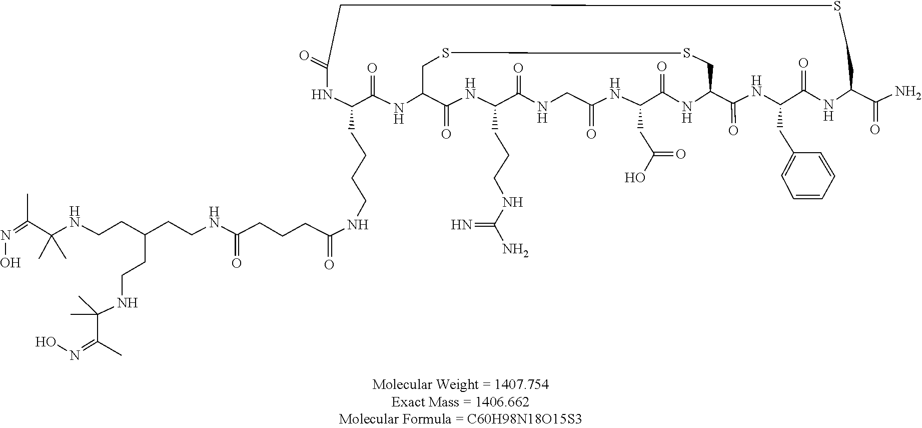 [SMILES: C/C(=N/O)C(C)(C)NCCC(CCNC(=O)CCCC(=O)NCCCC[C@@H]1NC(=O)CSC[C@@H](C(N)=O)NC(=O)[C@H](Cc2ccccc2)NC(=O)[C@@H]2CSSCC(NC1=O)C(=O)N[C@@H](CCCNC(=N)N)C(=O)NCC(=O)N[C@@H](CC(=O)O)C(=O)N2)CCNC(C)(C)/C(C)=N\O]

-NH₂, 9.1 mg of Chelator 1A (Example 5) and 6 μL of NMM was dissolved in DMF (0.5 ml). The mixture was stirred for 3 hours. Purification by preparative HPLC (Phenomenex Luna 5μ C18 (2) 250×21.20 mm column) of the reaction mixture was carried out using 0-30% B, where A=H₂O/0.1% TFA and B=CH₃CN/0.1% TFA, over 40 min at a flow rate of 10 ml/min. After lyophilisation 5.7 mg of pure material was obtained (Analytical HPLC: Gradient, 0-30% B over 10 min where A=H₂O/0.1% TFA and B=CH₃CN/0.1% TFA; column, Phenomenex Luna 3μ C18 (2) 50×4.6 mm; flow, 2 ml/min; detection, UV 214 nm; product retention time, 7.32 min). Further product characterisation was carried out using mass spectrometry: Expected, M+H at 1407.7, found, at 1407.6).

### Example 6: Synthesis of Disulfide [Cys²⁻⁶] Thioether Cyclo[CH₂CO-Lys(Chelator 1-glutaryl)-Cys²-Arg-Gly-Asp-Cys⁶-Phe-Cys]-(PEG)₃-NH₂ (Maraciclatide)

(Step 6a) Synthesis of 17-(Fmoc-amino)-5-oxo-6-aza-3,9,12,15-tetraoxaheptadecanoic acid.

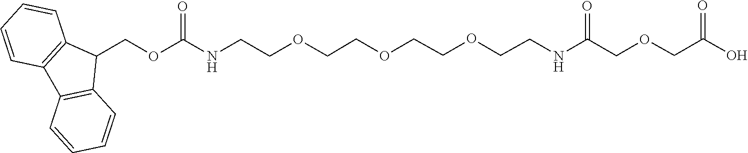 [SMILES: O=C(O)COCC(=O)NCCOCCOCCOCCNC(=O)OCC1c2ccccc2-c2ccccc21]

This building block is coupled to the solid-phase using Fmoc chemistry.

1,11-Diazido-3,6,9-trioxaundecane.

A solution of dry tetraethyleneglycol (19.4 g, 0.100 mol) and methanesulfonyl chloride (25.2 g, 0.220 mol) in dry THF (100 ml) was kept under argon and cooled to 0° C. in an ice/water bath. To the flask was added a solution of triethylamine (22.6 g, 0.220 mol) in dry THF (25 ml) dropwise over 45 min. After 1 hr the cooling bath was removed and stirring was continued for 4 hrs. Water (60 ml) was added. To the mixture was added sodium hydrogen carbonate (6 g, to pH 8) and sodium azide (14.3 g, 0.220 mmol), in that order. THF was removed by distillation and the aqueous solution was refluxed for 24 h (two layers formed). The mixture was cooled and ether (100 ml) was added. The aqueous phase was saturated with sodium chloride. The phases were separated and the aqueous phase was extracted with ether (4×50 ml). Combined organic phases were washed with brine (2×50 ml) and dried (MgSO₄). Filtration and concentration gave 22.1 g (91%) of yellow oil. The product was used in the next step without further purification.

11-Azido-3,6,9-trioxaundecanamine.

To a mechanically, vigorously stirred suspension of 1,11-diazido-3,6,9-trioxaundecane (20.8 g, 0.085 mol) in 5% hydrochloric acid (200 ml) was added a solution of triphenylphosphine (19.9 g, 0.073 mol) in ether (150 ml) over 3 hrs at room temperature. The reaction mixture was stirred for additional 24 hrs. The phases were separated and the aqueous phase was extracted with dichloromethane (3×40 ml). The aqueous phase was cooled in an ice/water bath and pH was adjusted to ca 12 by addition of KOH. The product was extracted into dichloromethane (5×50 ml). Combined organic phases were dried (MgSO₄). Filtration and evaporation gave 14.0 g (88%) of yellow oil. Analysis by MALDI-TOF mass spectroscopy (matrix: α-cyano-4-hydroxycinnamic acid) gave a M+H peak at 219 as expected. Further characterisation using ¹H (500 MHz) and ¹³C (125 MHz) NMR spectroscopy verified the structure.

17-Azido-5-oxo-6-aza-3,9,12,15-tetraoxaheptadecanoic acid.

To a solution of 11-azido-3,6,9-trioxaundecanamine (10.9 g, 50.0 mmol) in dichloromethane (100 ml) was added diglycolic anhydride (6.38 g, 55.0 mmol). The reaction mixture was stirred overnight. HPLC analysis (column Vydac 218TP54;solvents: A=water/0.1% TFA and B=acetonitrile/0.1% TFA; gradient 4-16% B over 20 min; flow 1.0 ml/min; UV detection at 214 and 284 nm), showed complete conversion of starting material to a product with retention time 18.3 min. The solution was concentrated to give quantitative yield of a yellow syrup. The product was analysed by LC-MS (ES ionisation) giving [MH]+ at 335 as expected. ¹H (500 MHz) and ¹³C (125 MHz) NMR spectroscopy was in agreement with structure The product was used in the next step without further purification.

17-Amino-5-oxo-6-aza-3,9,12,15-tetraoxaheptadecanoic acid.

A solution of 17-azido-5-oxo-6-aza-3,9,12,15-tetraoxaheptadecanoic acid (8.36 g, 25.0 mmol) in water (100 ml) was reduced using H₂(g)-Pd/C (10%). The reaction was run until LC-MS analysis showed complete conversion of starting material (column Vydac 218TP54; solvents: A=water/0.1% TFA and B=acetonitrile/0.1% TFA; gradient 4-16% B over 20 min; flow 1.0 ml/min; UV detection at 214 and 284 nm, ES ionisation giving M+H at 335 for starting material and 309 for the product). The solution was filtered and used directly in the next step.

17-(Fmoc-amino)-5-oxo-6-aza-3,9,12,15-tetraoxaheptadecanoic acid.

To the aqueous solution of 17-amino-5-oxo-6-aza-3,9,12,15-tetraoxaheptadecanoic acid from above (corresponding to 25.0 mmol amino acid) was added sodium bicarbonate (5.04 g, 60.0 mmol) and dioxan (40 ml). A solution of Fmoc-chloride (7.11 g, 0.275 mol) in dioxan (40 ml) was added dropwise. The reaction mixture was stirred overnight. Dioxan was evaporated off (rotavapor) and the aqueous phase was extracted with ethyl acetate. The aqueous phase was acidified by addition of hydrochloric acid and precipitated material was extracted into chloroform. The organic phase was dried (MgSO₄), filtered and concentrated to give 11.3 g (85%) of a yellow syrup. The structure was confirmed by LC-MS analysis (column Vydac 218TP54; solvents: A=water/0.1% TFA and B=acetonitrile/0.1% TFA; gradient 40-60% B over 20 min; flow 1.0 ml/min; UV detection at 214 and 254 nm, ES ionisation giving M+H at 531 as expected for the product peak at 5,8 minutes). The analysis showed very low content of side products and the material was used without further purification.

(Step 6b) Synthesis of ClCH₂CO-Lys-Cys(tBu)-Arg-Gly-Asp-Cys(tBu)-Phe-Cys-(PEG)₃-NH₂.

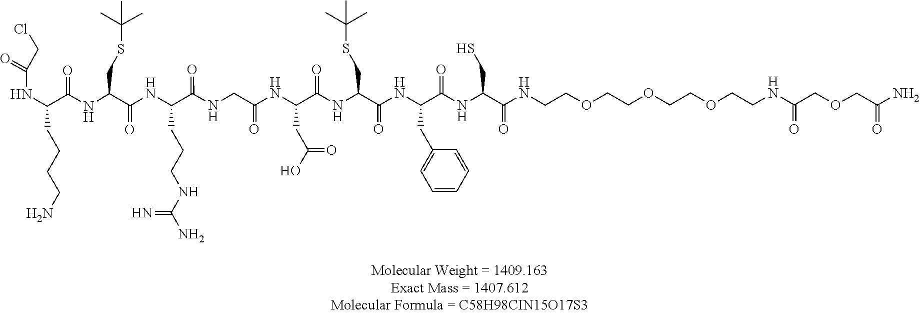 [SMILES: CC(C)(C)SC[C@H](NC(=O)[C@H](CCCCN)NC(=O)CCl)C(=O)N[C@@H](CCCNC(=N)N)C(=O)NCC(=O)N[C@@H](CC(=O)O)C(=O)N[C@@H](CSC(C)(C)C)C(=O)N[C@@H](Cc1ccccc1)C(=O)N[C@@H](CS)C(=O)NCCOCCOCCOCCNC(=O)COCC(N)=O]

The PEG unit was coupled manually to Rink Amide AM resin, starting on a 0.25 mmol scale, mediated by HATU activation. The remaining peptide was assembled on an ABI 433A automatic peptide synthesiser using 1 mmol amino acid cartridges. The amino acids were pre-activated using HBTU before coupling. N-terminal amine groups were chloroacetylated using a solution of chloroacetic anhydride in DMF for 30 min.

The simultaneous removal of peptide and side-chain protecting groups (except tBu) from the resin was carried out in TFA containing TIS (5%), H₂O (5%) and phenol (2.5%) for two hours. After work-up 322 mg of crude peptide was obtained (Analytical HPLC: Gradient, 5-50% B over 10 min where A=H₂O/0.1% TFA and B=CH₃CN/0.1% TFA; column, Phenomenex Luna 3μ C18 (2) 50×4.6 mm; flow, 2 ml/min; detection, UV 214 nm; product retention time, 6.37 min). Further product characterisation was carried out using mass spectrometry: Expected, M+H at 1409, found, at 1415).

(Step 6c) Synthesis of Thioether Cyclo[CH₂CO-Lys-Cys(tBu)-Arg-Gly-Asp-Cys(tBu)-Phe-Cys]-(PEG)₃-NH₂ (NC100717)

See Indrevoll et al [Bioorg.Med.Chem.Lett., 16, 6190-6193 (2006)].

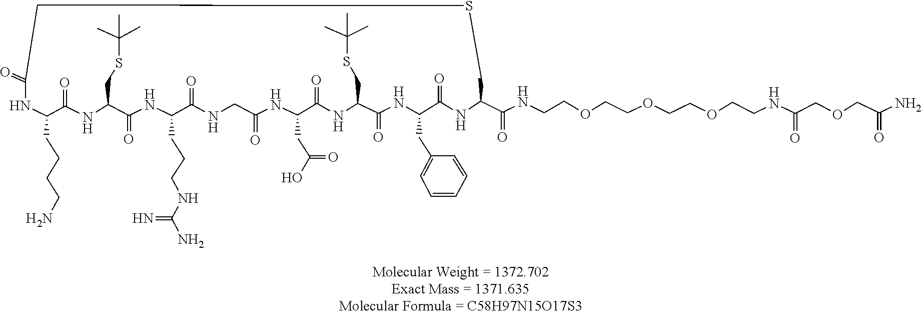 [SMILES: CC(C)(C)SC[C@@H]1NC(=O)[C@H](CCCCN)NC(=O)CSC[C@@H](C(=O)NCCOCCOCCOCCNC(=O)COCC(N)=O)NC(=O)[C@H](Cc2ccccc2)NC(=O)[C@H](CSC(C)(C)C)NC(=O)[C@H](CC(=O)O)NC(=O)CNC(=O)[C@H](CCCNC(=N)N)NC1=O]

322 mg of ClCH₂CO-Lys-Cys(tBu)-Arg-Gly-Asp-Cys(tBu)-Phe-Cys-(PEG)₃-NH₂ was dissolved in water/acetonitrile. The mixture was adjusted to pH 8 with ammonia solution and stirred for 16 hours.

After work-up, crude peptide was obtained (Analytical HPLC: Gradient, 5-50% B over 10 min where A=H₂O/0.1% TFA and B=CH₃CN/0.1% TFA; column, Phenomenex Luna 3μ C18 (2) 50×4.6 mm; flow, 2 ml/min; detection, UV 214 nm; product retention time, 6.22 min). Further product characterisation was carried out using mass spectrometry: Expected, M+H at 1373, found, at 1378).

(Step 6d) Synthesis of Disulfide [Cys²⁻⁶] Thioether Cyclo[CH₂CO-Lys-Cys²-Arg-Gly-Asp-Cys⁶-Phe-Cys]-(PEG)₃-NH₂.

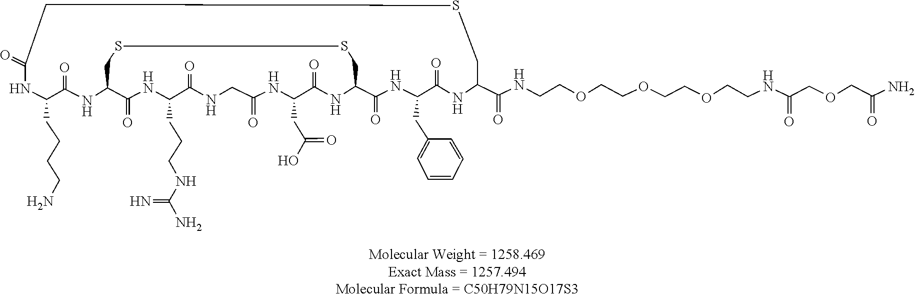

Thioether cyclo[CH₂CO-Lys-Cys(tBu)-Arg-Gly-Asp-Cys(tBu)-Phe-Cys]-(PEG)₃-NH₂ was treated with a solution of anisole (200 μL), DMSO (2 ml) and TFA (100 ml) for 60 min following which the TFA was removed in vacuo and the peptide precipitated by the addition of diethyl ether. Purification by preparative HPLC (Phenomenex Luna 5μ C18 (2) 250×21.20 mm column) of 70 mg crude material was carried out using 0-30% B, where A=H₂O/0.1% TFA and B=CH₃CN/0.1% TFA, over 40 min at a flow rate of 10 ml/min. After lyophilisation 46 mg of pure material was obtained (Analytical HPLC: Gradient, 0-30% B over 10 min where A=H₂O/0.1% TFA and B=CH₃CN/0.1% TFA; column, Phenomenex Luna 3μ C18 (2) 50×4.6 mm; flow, 2 ml/min; detection, UV 214 nm; product retention time, 6.80 min). Further product characterisation was carried out using mass spectrometry: Expected, M+H at 1258.5, found, at 1258.8).

(Step 6e) Synthesis of Disulfide [Cys²⁻⁶] Thioether Cyclo[CH₂CO-Lys(Chelator 1-glutaryl)-Cys²-Arg-Gly-Asp-Cys⁶-Phe-Cys]-(PEG)₃-NH₂.

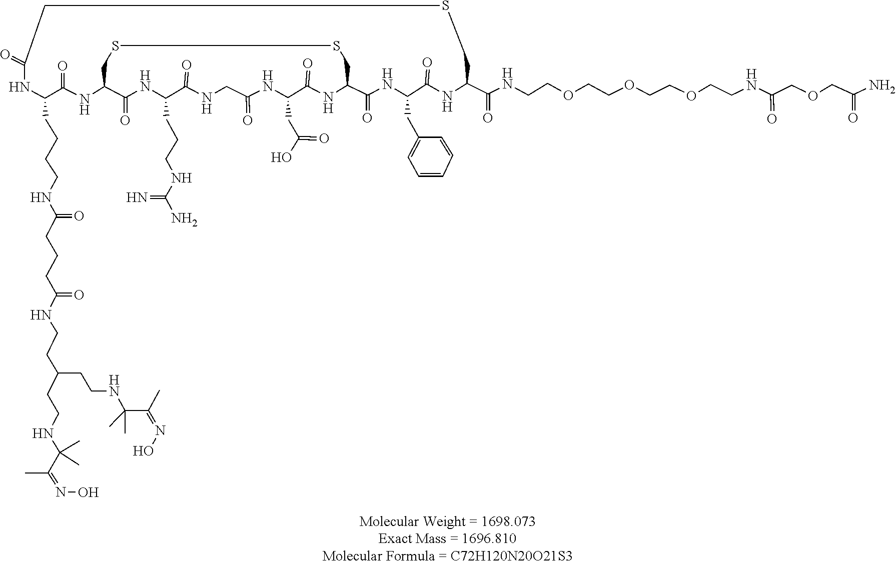

13 mg of [Cys²⁻⁶] cyclo[CH₂CO-Lys-Cys-Arg-Gly-Asp-Cys-Phe-Cys]-(PEG)₃-NH₂, 9.6 mg of Chelator 1A and 8 μL of NMM was dissolved in DMF (0.5 ml). The mixture was stirred for 2 hours and 30 minutes. Purification by preparative HPLC (Phenomenex Luna 5μ C18 (2) 250×21.20 mm column) of the reaction mixture was carried out using 0-30% B, where A=H₂O/0.1% TFA and B=CH₃CN/0.1% TFA, over 40 min at a flow rate of 10 ml/min. After lyophilisation 14.2 mg of pure material was obtained (Analytical HPLC: Gradient, 0-30% B over 10 min where A=H₂O/0.1% TFA and B=CH₃CN/0.1% TFA; column, Phenomenex Luna 3μ C18 (2) 50×4.6 mm; flow, 2 ml/min; detection, UV 214 nm; product retention time, 7.87 min). Further product characterisation was carried out using mass spectrometry: Expected, M+H at 1697.8, found, at 1697.9).

### Example 7: Preparation of Maraciclatide Lyophilised Non-Radioactive Kit

A lyophilised kit was prepared having the following formulation:

| Component | Quantity per vial (μg) |
|---|---|
| Maraciclatide | 75 |
| Stannous chloride dihydrate | 17.8 |
| Methylene diphosphonic acid sodium salt | 90 |
| Para-amino-benzoic acid (pABA) sodium salt | 200 |
| Sodium hydrogen carbonate | 1800 |
| Sodium carbonate anhydrous | 630 |

The Maraciclatide and excipients are dissolved in water for injection under a nitrogen atmosphere. Aliquots are dispensed into glass vials after sterile filtration and a synthetic rubber closure added. The solution is then frozen at −42° C. and lyophilised under vacuum to remove the water. The lyophilised material is then closed under a nitrogen atmosphere and sealed with an aluminium overseal. The vials may be stored at 2-8° C. for an extended period before use.

### Example 8: Preparation of ^{99m}Tc-Maraciclatide

The kit of Example 7 was reconstituted with generator eluate of up to 3.1 GBq/6 ml from a commercial Technetium Generator under aseptic conditions and allowed to stand at room temperature for 20 minutes. Quality Control is performed by both visual assessment and measurement of radiochemical purity (RCP) with thin layer chromatograph using silica coated paper and a mobile phase comprising 50:50 methanol and 1 molar ammonium acetate. The reconstituted solution is colourless and free from visible particles with RCP greater than 85%. Sufficient material to provide a patient dose is withdrawn into a syringe under aseptic conditions.

### Example 9: Uptake in Animal Models of Endometriosis

A rat surgical model of endometriosis was used, together with immunocompromised mouse and isogenic mouse models—all 3 animal models of endometriosis are as described by Story, L. ILAR J., 45(2) 132-138 (2004).

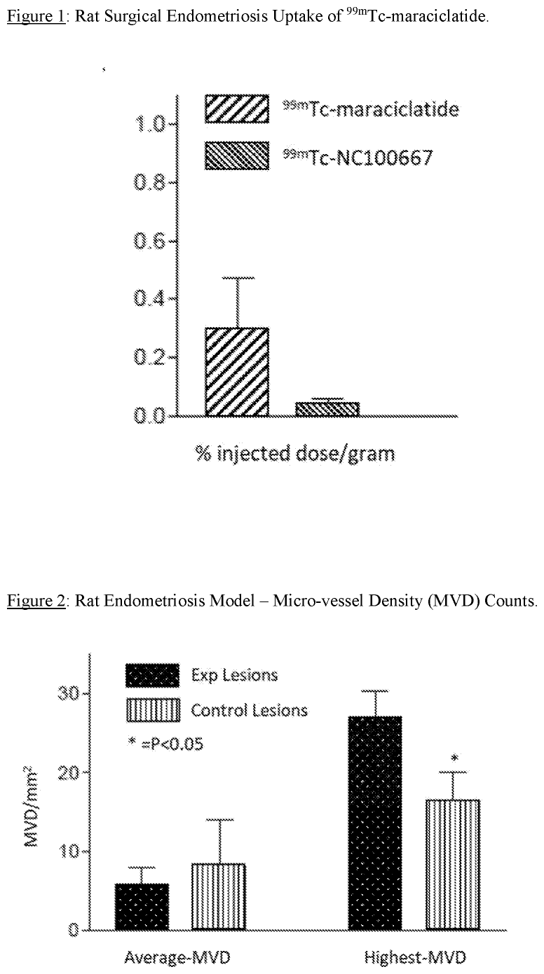

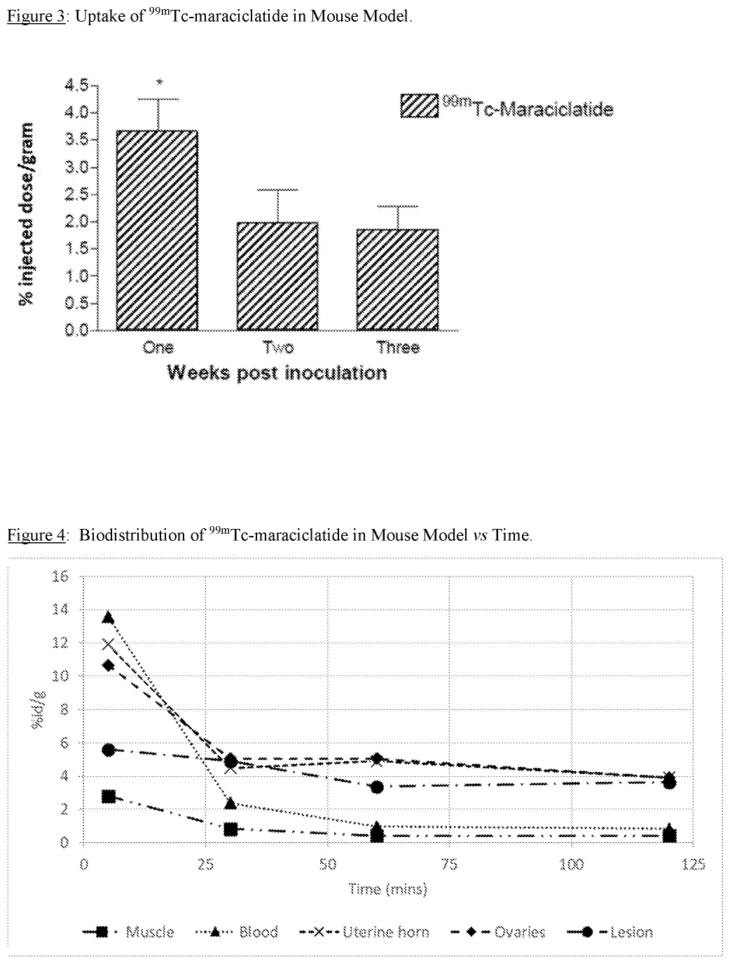

The uptake of ^{99m}Tc-maraciclatide was compared with that of ^{99m}Tc-NC100677. NC100667 is described by Edwards et al [D. Edwards, Nucl.Med.Biol., 35, 365-375 (2008)]. NC100667 is a negative control peptide, in which the arginine residue of the RGD binding motif of maraciclatide (NC100692) was replaced with an alanine residue. Thus, only one amino acid residue was changed—all other aspects of the agent were otherwise the same. The results are shown in FIGS. 1 to 4. Uptake and retention of ^{99m}Tc-maraciclatide was demonstrated in all experimental animal models of endometriosis.

1. A method of imaging site(s) of endometriosis in a subject, which comprises administering the radiopharmaceutical ^{99m}Tc-maraciclatide to said subject, followed by extra-uterine imaging of the radioactive emissions from said ^{99m}Tc-maraciclatide, wherein said site(s) comprise one or more of the following:
(i) abdominal endometriosis;
(ii) peritoneal endometriosis; or
(iii) thoracic endometriosis.

2. The method of claim 1, where said site(s) of endometriosis comprise superficial endometriotic lesions.

3. The method of claim 1, where the site(s) of endometriosis comprise abdominal or peritoneal endometriosis.

4. The method of claim 1, where superficial endometriotic lesions are imaged in combination with peritoneal endometriosis,

5. The method of claim 1, where all 3 of (i)-(iii) are imaged via whole body imaging of said subject.

6. The method of claim 1, where the radioactive emissions are detected and processed using: a gamma camera; a gamma detector; image processing software or a combination thereof.

7. The method of claim 6, where the gamma camera is a high resolution gamma camera having a spatial resolution of less than 1 millimetre.

8. The method of claim 7, where the high resolution gamma camera comprises a CZT detector.

9. The method of claim 1, further comprising preparing the radiopharmaceutical ^{99m}Tc-maraciclatide by reacting maraciclatide with ^{99m}Tc-pertechnetate in the presence of a reducing agent.

10. The method of claim 1, further comprising preparing the ^{99m}Tc-maraciclatide radiopharmaceutical by reconstituting a non-radioactive kit with a solution comprising ^{99m}Tc-pertechnetate, wherein said non-radioactive kit comprises:
a) maraciclatide;
b) para-aminobenzoic acid or a salt thereof with a biocompatible cation;
c) a stannous reductant;
d) methylene diphosphonic acid or a salt thereof with a biocompatible cation.

11. A method of diagnosing endometriosis in a subject which comprises the method of claim 1.

12. A method of treating a subject diagnosed with endometriosis, comprising the method of imaging of claim 1 to identify said subject, and treating said subject with a therapy for endometriosis.

13. A method of monitoring a subject undergoing therapy for endometriosis, comprising providing therapy to the subject, following by imaging the subject by the method according to claim 1.

14-15. (canceled)
